(19) United States
(12) Patent Application Publication
(10) Pub. No.: US 2007/0196368 A2
(43) Pub. Date: Aug. 23, 2007

(54) Method for Controlled Induction of Somatic Mutations and Use Thereof in Proteomics

(75) Inventors: Claude-Agnes Reynaud, Paris (FR); Jean-Claude Weill, Paris (FR)

Correspondence Address: Marina Larson & Associates, LLC, P.O. BOX 4928, DILLON, CO, 80435, US

(73) Assignee: Mixis France, SA, Paris (FR)

(21) Appl. No.: 10/499,398

(22) Filed: Oct. 18, 2004

(30) Foreign Application Priority Data
Dec. 18, 2001 (FR) 01/16352
Aug. 8, 2002 (FR) 02/100078

(51) Int. Cl. A61K 39/395; C12N 15/85

(52) U.S. Cl. 424/144.1; 435/455

(57) ABSTRACT

The invention concerns a method for inducing somatic mutations in vitro or ex vivo, comprising (1): adding to the cells to be mutated, in culture conditions and in a medium suited to said cells, of at least a combination of anti-IgM and anti-CD19 and/or anti-DC21 antibodies, while (2) said anti-IgM antibodies are biotinylated and are subjected to specific aggregation by streptavidin coupled with a support. Advantageously, the mutations are controlled and/or adjusted by inhibiting and reactivating and/or stimulating DNA polymerase pol iota. The invention is in particular applicable to rapid and controlled induction of mutations on the BL2 Burkitt's lymphoma.

### FIELD OF THE INVENTION

The present invention relates to biology, and more specifically to the field of adaptation by directed mutation, specific to the living world. It relates more particularly to in vitro induction of somatic mutations, as well as to the applications henceforth made possible by improvements of such a technique.

In a particular embodiment, the invention relates to the induction of mutations in BL2 Burkitt's lymphoma cells.

### TECHNOLOGICAL BACKGROUND OF THE INVENTION

To simplify matters, reference is made below to BL2 Burkitt's lymphoma. However, it should be stressed that this is done only to facilitate the discussion of the technique in question and of the inventive concept claimed, without in any way limiting the scope of the invention.

The type I BL2 Burkitt's lymphoma cell line exhibits the characteristics of the centroblastic B cells. This cell line is the closest immortalized counterpart of the centroblasts of germinal centers. Its germinal-center origin has been confirmed by the presence of somatic mutations in the immunoglobulin V_{H} genes of Burkitt's lymphomas.

The homogeneity and stability of BL2 cells in suitable cultures have been stressed (by Denépoux, S. et al., Immunity, vol. 6, 35-46, 1997). It has been proposed, in conformity with the centroblast phenotype, to consider that BL2 results from the transformation of a germinal-center centroblast which has undergone several somatic mutation phases.

Moreover, it is considered that B cells undergo hypermutation at the centroblast stage (Pascual, V. et al., J. Exp. Med. 180, 329-339, 1994).

The phenomenon of immunoglobulin hypermutation takes place in the germinal centers, after stimulation of a B cell by a T-dependent antigen.

In particular, Denépoux, S. et al. (op. cit.) have established that lymphoma cells, notably the BL2 cells, can trigger a hypermutation process after aggregation of their surface receptor and co-culture with an auxiliary or amplifying T cell, after a week of culture. It was concluded in this article that it is by repeated activations of BL2 cells that the mutation frequency can be increased. These authors have also emphasized that the mutations induced in vitro in the BL2 cell do not affect the constant IgM region, and do not cause the appearance of an antigen-directed selection.

Likewise, Yélamos, J. et al., Nature, vol. 376, pp. 225-229 (1995), have reported on experiments which tend to show that the V gene fragment of the immunoglobulin is not indispensable for a hypermutation, and that the construction of artificial mutation substrates could turn out to be simplified.

Despite these developments, so far there are no effective means available for investigating these phenomena and for deriving practical results from such studies for diagnostic and/or follow-up purposes, as well as for the treatment of tumoral processes under practical conditions and with satisfactory speed.

Hence, a need exists for finding means which make it possible to carry out rapid and reliable somatic hypermutation tests. More particularly, there is a need for a model of somatic mutation induction applicable to V genes, as well as to other genes that could be implicated in a tumoral process, particularly in humans, such as, for example, the Bcl-6 and Fas Ligand (FasL) genes.

It has now been found that lymphoma cells, notably the BL2 cells, can trigger an accelerated hypermutation process, in contradiction to prior-art data which indicated that a comparable process took a long time and was tedious to carry out.

More precisely, it has now been unexpectedly found that, with the BL2 cell as model, it is possible, in conditions of exceptional speed and efficiency, to carry out the induction of somatic mutations of appropriate cells in vitro, by adding to the cells to be mutated, under suitable culturing conditions and in a medium suitable for said cells, at least one ternary combination of anti-CD19, anti-CD21 and anti-IgM antibodies.

Thus, the invention first relates to a process for in vitro or ex vivo induction of somatic mutations, comprising (1) the addition, to the cells to be mutated, and under suitable culturing conditions and in a medium that is suitable for said cells, of at least one ternary combination of anti-CD19, anti-CD21 and anti-IgM antibodies, while (2) said anti-IgM antibodies are biotinylated and are submitted to specific aggregation by streptavidin coupled to a support, advantageously to a support consisting entirely or in part of small magnetic balls.

According to one characteristic, the induction of somatic mutations according to the invention is carried out over a short period, the duration of the process being, in practice, of the order of 1 hour 30 minutes (one and a half hours).

According to an advantageous characteristic, the process according to the invention does not comprise the use of co-cultures with cells of another cell type, notably with a T cell.

It has, in fact, been observed that the process recommended according to the invention, in which a specific aggregation of biotinylated anti-IgM antibodies by streptavidin coupled to a support such as, preferably, small magnetic balls, is carried out, as well as a combination of at least the three anti-CD19, anti-CD21 and anti-IgM antibodies, causes a rapid in vitro mutation of cells that are cultured in the presence of these antibodies, achieving this in a very short time which has no relationship to the durations indicated for obtaining in vitro mutations according to the prior-art technique.

According to one variant, the process according to the invention may comprise a combination of anti-IgM antibodies selected from a combination of anti-IgM and anti-CD19 antibodies, a combination of anti-IgM and anti-CD21 antibodies, and their mixtures in all proportions.

According to another characteristic, the process according to the invention is used for inducing mutations in BL2 Burkitt's lymphoma cells.

The invention also relates to the use of this process of induction of somatic mutations for carrying out in vitro hypermutagenicity tests at the protein and/or gene level.

According to one embodiment, this use is applied to B lymphoma cells, in particular to human lymphoma B cells, and more particularly for the induction of mutations in BL2 Burkitt's lymphoma cells.

According to one particularly preferred embodiment, the system according to the invention is used for induction of mutations in BL2 Burkitt's lymphoma cells in the G0/G1 phase.

The invention also relates to a kit for induction of somatic mutations, characterized in that it comprises a somatic mutation-inducing system such as defined above.

Another object of the invention is the use of said kit for qualitative and/or quantitative identification of components of the mutasome, in particular by protein analysis.

In one embodiment, the use of the kit according to the invention comprises identification of the induced post-translational modifications, in particular by identification, isolation and analysis of a component of the mutasome, particularly of a protein component, which appears during said induction.

According to a preferred characteristic of using this process, said use comprises providing at least one gene, in particular a gene coding for a protein of interest, in which it is desired to induce mutations, while said gene is included in a cassette containing the promoter and activator (enhancer) of the genes coding for the heavy or light chain of the IgG's, and said gene is transfected in the lymphoma cells, in particular in BL2 Burkitt's lymphoma cells.

According to an advantageous form of carrying out the invention in order to bring about mutations in any sequence, the sequence to be mutated is surrounded by the promoter and the activator of the Ig's, preferably in a mutation cassette.

The invention is described below by referring to one embodiment, which is purely illustrative and whose purpose is to provide a person skilled in the art with practical indications for carrying out the invention. On the basis of these indications and his own knowledge, a person skilled in the art is able, without exceeding the scope of the present invention, to conceive of analogous or equivalent test systems and different concrete uses, whether or not related to those that are described herein.

For in vitro induction of somatic mutations in the BL2 cell, the following procedure was used:

Culturing Conditions of the BL2 Cell

BL2 cells were provided and maintained in a complete medium at the rate of 10⁵ cells per mL for 24 hours at 37° C. and at 5% CO₂.

The culture medium was RPMI 1640 completed with 10% FCS, as well as 100 U/mL (units/mL) of penicillin and 100 μg/mL of streptomycin.

Stimulation

To remove all traces of serum, the cells were washed in RPMI medium by centrifugation at 1500 rpm for 7 minutes at a temperature of between 4 and 10° C.

The BL2 cells were taken up in RPMI medium at the rate of 10⁶ cells per 500 μL. 10⁶ cells were introduced in ice in a 15 mL flask, and the following were added:
20 μL of anti-CD19 coupled to FITC (provided by Immunotech),
20 μL of anti-CD21 coupled to PE (provided by Becton Dickinson) and
4 μL of anti-IgM coupled to biotin (provided by Immunotech).

After shaking until a homogeneous mixture was obtained, the latter was placed on ice and left standing for about 20 minutes.

The cells of this mixture were then washed in 10 mL of RPMI medium (at 4° C.), then subjected to centrifugation at 1500 rpm for 7 minutes. The cells washed in this manner were taken up in 500 μL of RPMI in an Eppendorf tube (1.5 mL), and 20 μL of small magnetic balls (commercially available under the name Dynal) to which streptavidin was coupled, were added. The tube was turned round on a wheel at about 4° C. for 15 minutes.

The cells and the small balls to which said cells were fixed were taken up in about 4 μL of complete medium to which ADCM (provided by the Centre de transfusion de Lyon, France [Transfusion Center of Lyon, France]) was added, and the cells thus treated were distributed on a titration plate comprising 24 wells for cell culture (at the rate of 2×10⁵ cells per well).

The cells were left to incubate for about 1 hour 30 minutes at 37° C. The entire content of a well was taken up in a 1.5 mL Eppendorf tube and centrifuged at 2000 rpm for 10 minutes.

The product of centrifugation was recovered in order to remove the supernatant and plunge the centrifugation sediment into liquid nitrogen.

The mutations were tested in DNA by conventional molecular techniques that are known and currently practiced by persons skilled in the art.

Analysis of the obtained results and of those of the investigations concurrently carried out on genes different from V_{H} and/or with cells other than BL2, shows that it is possible in this way to induce in vitro mutations in the BL2 cell on the V_{H} gene rearranged by stimulation of a group of surface receptors.

As a variant, the Ig sequence can be replaced, e.g., by a prokaryote sequence, such as the neomycin-resisting gene, and obtain similar mutations with BL2.

It could thus be established, according to the invention, that:
mutations can be induced in about 1 hour 30 minutes after stimulation by the antigen receptor and a group of co-receptors, in the absence of co-culture with T cells;
about one third of the cells, on average, undergo the hypermutation mechanism;
the mutation induced according to the invention takes place in the presence of actinomycin D, which totally inhibits the transcription;
the mutations are induced when the cells are maintained in G0/G1 by hydroxyurea;
the mutations are also induced in about 30 minutes when the cells are prepared in phase G0/G1 by elutriation.

These results run counter to the teachings of the prior art, and show that the hypermutation process can take place in the absence of transcription and sister chromatid.

It is thus possible, in practice, to study, on two-dimensional gel, the loss or appearance of proteins, as well as their post-translational modification.

Moreover, somatic mutations realized according to the present invention could be reinduced in suitable cells such as BL2 Burkitt's lymphoma cells by stimulation of the surface receptors. A stimulation of this type very probably takes places in vivo as well.

The process and means of the invention make it possible to approach the studies and tests of determination of hypermutation phenomena of genes—in particular of immunoglobulin genes—in a radically more effective and reliable manner.

Certain developments and applications that may be taken into consideration by using the means of the invention are summarized below. It is clear that this list is not limitative and that other applications can also be considered.

By way of example, it is important that, according to the invention, it is now possible to determine, under conditions that could not be realized until now, the role and importance of somatic hypermutation in the malignant transformation of B cells in the germinal center.

By providing a unique model of simplicity and rapidity of induction of somatic mutations, the invention has made it possible to show that the hypermutation process can take place in the absence of transcription and of sister chromatid.

It also provides means for obtaining the hypermutation signature at the protein and genic level, so as to make available a simple test that is applicable to human B lymphomas, in particular.

Other questions not so far resolved are, in particular, that of the molecular mechanism of hypermutation and of the possible presence of a highly mutagenic polymerase, as well as that of the cofactors present.

In effect, it should be recalled that the cofactors, i.e., the components of the mutasome which permit the targeting of the hypermutation process on the V_{H} and V_{L} genes rearranged at a precise stage of the immune response, are not known. The test system of the invention provides reliable and rapid means with which to follow the kinetics of such a hypermutation process and to enable a person skilled in the art to carry out, on the basis of his own knowledge and the information provided by the present description, rapid and economical tests that are applicable to human B lymphomas in particular.

Thus, the invention further relates to a kit or set of means for the induction of hypermutations and for carrying out tests relating thereto, wherein the means for the induction of a rapid hypermutation comprises a combination of antibodies such as described above.

In a more general way, the present invention provides means which permit the study and follow-up of the first molecular stages of somatic mutation processes at the DNA and protein level.

In short, it is thus possible, by using the means according to the invention, to modify a gene by homologous recombination or to inactivate it, for example in the BL2 cell. The technique according to the invention which is thus made realizable in the cells, in particular in BL2 cells, makes it possible to improve the mutator properties of these cells, and also to test the role which this or that molecule could play in the physiology of B cells and of Burkitt's lymphomas.

Hence, by using the means according to the invention, it is possible, by way of norilirnitative examples, to obtain overexpression of certain genes in BL2 and thereby improve the latter's mutator cell performances, whereas transfection of the molecules which seem implicated in the process may concern the following, among others: AID, MSH2, MSH6 or enzymes of type RAD30A and/or RAD30B.

The mutations are induced in the G1 phase of the cell cycle and are produced on a DNA strand of the rearranged V_{H} gene, with possible fixation by replication in one of the daughter cells.

Furthermore, the present inventors were also able to establish the determinant role of Pol iota—which is a DNA polymerase of the Y family—in such a somatic hypermutation process (SHM) of the immunoglobulin genes.

To produce BL2 clones deficient in Pol iota (which is one of the two human homologues of the yeast RAD30 showing a highly error-prone polymerase activity of filling the short DNA gaps), a process of gene inactivation similar to that which has been developed for inactivation of the AID gene (AID: activation-induced cytidine deaminase) was used. The AID molecule has been described, in particular, by Muramatsu, M. et al., “Class switch recombination and hypermutation require activation-induced cytidine deaminase (AID), a potential RNA editing enzyme,” Cell 102, 553-63 (2000); and by Revy, P. et al., “Activation-induced cytidine deaminase (AID) deficiency causes the autosomal recessive form of the Hyper-IgM syndrome (HIGM2),” Cell 102, 565-75 (2000).

Two Pol iota^{-/-} clones (54 and 267) were generated from a Pol iota heterozygote clone. Appropriate constructions were used to inactivate the two alleles. The two iota^{-/-} clones had the same proliferation rate as the original BL2 cell line, with a similar mitotic index and cell cycle profile, and showed no chromosomal aberrations or translocations. No Pol iota expression was detected in the Western blots of cell extracts of iota^{-/-} clones 54 and 267 when using high rabbit polyclonal antibodies against human Pol iota. The Pol iota expression was restored by transfection of pIRES vectors ordering the expression of complete human Pol iota cDNA under the control of the CMV promoter; in the experiments carried out, the rates of expression were comparable to that noted in of normal BL2 cell lines, within the limits of variation inherent in Western blot quantifications. After several attempts, it was not possible to obtain clones over-expressing Pol iota, either on a normal background or on a background deficient in Pol iota. To continue the analysis, two restored clones were selected for each target cell.

Two different methods can be used for inducing, in the BL2 cell line, a hypermutation of its rearranged V_{H} gene. The first method involves cross linkage of anti-IgM in co-culture with an auxiliary T clone, or a line of auxiliary T cells, in which case the mutations, if they are produced, are observed after 3 days of culture (see Denépoux, S. et al., supra; and Poltoratsky, V. et al., [in English:] “Expression of error-prone polymerases in BL2 cells activated for Ig somatic hypermutation,” Proc. Natl. Acad. Sci. USA 98, 7676-81 (2001)). In the present test, this SHM induction system was adapted by use of the cell line of CB15, which is an auxiliary T cell transformed by the Saimiri herpes virus, in co-culture, and IgM cross linkage followed by aggregation of the biotinylated anti-IgM antibody by small streptavidin balls. The second method only involves cross linking of the three surface receptors IgM, CD19 and CD21, or of a combination of IgM+CD19 and/or IgM+CD21, followed by the same aggregation of the biotinylated anti-IgM antibody by small streptavidin balls, in which case mutations were observed after only 90 minutes of incubation. The mutation frequency resulting from a single round of mutagenesis was similar for both methods, i.e., 4-6×10⁻⁴ per base pair (see Table 1 below). The BL2 cell line also shows a very low frequency of constitutive mutation of its rearranged V_{H} gene when it is maintained in culture, which practically does not exceed 10⁻⁴ mutations per base pair after 2 to 3 months of culture.

No detectable increase in mutation frequency was observed when the clones deficient in Pol iota 54 or 267 were induced for hypermutation, both in the culture of T cells and in the test with the aforementioned three antibodies: only 7 mutations in 298 V sequences were recorded after stimulation as against 5 in 222 sequences before stimulation. By contrast, the induction of mutations was restored by re-expression of Pol iota cDNA in these clones at levels comparable to those shown by the normal BL2: 83 mutations in 547 V sequences after stimulation, as against 6 in 547 V sequences before stimulation. Moreover, the restored mutation scheme in these clones expressing Pol iota was similar to the mutation scheme of the BL2, with a high targeting of the GC base pairs and a tendency for transitions (see Table 2). As for the normal BL2, the Cμ gene of the restored Pol iota clones did not appear to be targeted for the mutation after induction.

TABLE 1
V gene mutation frequencies in subclones of BL2
deficient in Pol iota and competent for Pol iota

| | | | Stimulated | | | |
|---|---|---|---|---|---|---|
| Type of Cell | Not stimulated | | Anti-IgM/CD19/CD21 | | Anti-IgM/CB15 | |
| BL2 | 0.71 × 10⁻⁴ | (7/236) | 3.7 × 10⁻⁴ | (29/187) | 3.8 × 10⁻⁴ | (26/165) |

Pol iota^{-/-} clones

| | | | | | | |
|---|---|---|---|---|---|---|
| 54 | 0.82 × 10⁻⁴ | (3/88) | 0.96 × 10⁻⁴ | (4/100) | 0.51 × 10⁻⁴ | (2/95) |
| 267 | 0.36 × 10⁻⁴ | (2/134) | 1.00 × 10⁻⁴ | (1/24) | <0.30 × 10⁻⁴ | (0/79) |

Pol iota^{-/-} clones restored with Pol iota cDNA

| 54-7 | 0.31 × 10⁻⁴ | (1/78) | 2.9 × 10⁻⁴ | (14/115) | 3.1 × 10⁻⁴ | (9/69) |
|---|---|---|---|---|---|---|
| 54-10 | 0.43 × 10⁻⁴ | (2/111) | 2.4 × 10⁻⁴ | (8/79) | n.d. | |
| 267-20 | 0.27 × 10⁻⁴ | (1/89) | 4.0 × 10⁻⁴ | (19/113) | 5.1 × 10⁻⁴ | (22/104) |

| | | | | | |
|---|---|---|---|---|---|
| 267-12 | 0.50 × 10⁻⁴ | (2/97) | 4.0 × 10⁻⁴ | (11/67) | n.d. |

Note: Mutation frequencies are expressed as mutation per nucleotide, with the total number of mutations per sequenced V gene (416 bp) indicated between parentheses.

TABLE 2
Percentage of substitution of nucleotides in mutations generated in the V4-39-JH5 gene
of the Pol iota^{-/-} BL2 cell line of B2 restored for Pol iota^{a} expression

| | To: | | | | | | | |
|---|---|---|---|---|---|---|---|---|
| From: | A | | T | | G | | C | Total |
| A | | | 3.6 | (0.9) | 7.2 | (4.6) | 1.8 (1.4) | 24.9 (20.5) |
| T | 4.6 | (3.1) | | | 0 | (2.7) | 7.7 (7.8) | |
| G | 28.1 | (26.3) | 13.5 | (4.8) | | | 1.3 (7.5) | 75.1 (79.5) |
| C | 9.6 | (10.6) | 19.0 | (24.2) | 3.6 | (6.1) | | |

| | | | | |
|---|---|---|---|---|
| Transitions: | 62.0 | (62.9) | Transversions: 38.0 | (37.1) |

Note: Seventy-four substitutions of targeted bases in 416 bp of the VDJ sequence were analyzed out of a total of 83 mutations comprising 9 insertions (indicated in Table 1). The percentage of substitution is corrected for the composition of bases of the V segment. The numbers in bold print relate to the mutations induced in the restored Pol iota clones, while the values relating to the induction of normal BL2 are between parentheses.

Another subject of the invention is the use of Pol iota DNA polymerase for adjustment of the hypermutagenicity in in vitro hypermutagenicity tests at the protein and/or genic level, in particular on B lymphomas, and particularly on BL2 Burkitt's lymphoma.

Another subject of the invention is the use of an improved Pol iota DNA polymerase, particularly of one restored after change of at least one of its natural amino acids according to techniques known to persons skilled in the art to increase the hypermutagenicity in such tests. According to the invention, this improvement of Pol iota is advantageously carried out by random mutagenesis of the annular domain II (“ring finger”) with a view to replacing at least one amino acid therein, followed by restoration of the functionalities of the enzyme.

The invention further relates to a kit for inducing somatic mutations such as described above, and which additionally comprises means for inactivation of the Pol iota gene and/or for the latter's restoration by Pol iota cDNA expression.

Identification of this Pol iota contribution is illustrated in greater detail in the experimental part below.

Induction of Hypermutation of the V Gene in the BL2 Cell Line:

Hypermutation of the V gene was induced according to two different modes of operation. In using the first mode of operation, cross linkages of the surface receptors IgM, CD19 and CD21 were effected in accordance with the protocol described above. The second mode of activation was based on a co-culture with the CB15 cell line, which is a line of auxiliary T cells transformed by the Saimiri herpes virus. The CB15 was cultured in RPMI 1640 medium (Invitrogen), completed with 10% fetal calf serum (Hyclone), penicillin/streptomycin (Invitrogen) and 50 units/mL of IL2 (Sigma). The CB15 was irradiated at 4000 rads, resuspended in complete medium plus 2% Caryoser (CTS, Lyon), and deposited at the rate of 500,000 cells per well in plates having 24 wells or alveoles which had been coated during one night with anti-CD3 (OKT3, Janssen-Cilag, 2.5 μg/well). 2×10⁶ BL2 cells were incubated in 0.5 mL of RPMI for 20 minutes on ice with a biotinylated anti-IgM antibody (0.6 mg/mL, Caltag Laboratories), then crosslinked with small magnetic balls conjugated with streptavidin (Dynabeads M280, Dynal) for 20 minutes on a rotating plate and at low temperature. The BL2 was resuspended at the rate of 100,000 cells per mL in RPMI medium completed with 2% Caryoser, and 0.5 mL per well was deposited on a plate of 24 wells containing irradiated CB15 and anti-CD3 (in a BL2: CB15 ratio of 1:10). The cells were removed after 3 days of incubation at 37° C., the DNA was extracted by means of an appropriate kit called DNeasy tissue kit (Qiagen), and the mutations were analyzed after amplification of the V4-39-JH5 gene. The mutations were identified by multiple alignments of electropherograms and use of the AutoAssembler software (Applera).

Inactivation of the Pol Iota Gene in the BL2:

The genomic sequence of human Pol iota was collected from the HTGS clone of 180 kb AC021325, which contains all the coding exons. The DNA fragments used to generate the targeting constructions were amplified from BL2 genomic DNA and cloned first with the cloning kit for ACP TOPO-XL (Invitrogen), excised by means of Sall and Xhol if their sites had been added to the ACP primers, or in the form of Mlul-Notl fragments if restriction sites were not included, and then cloned in the corresponding sites of a pBSK vector (Stratagene) to which the Sfil and Mlul sites had been added at the Sacl site of its polylinker. Genes resistant to neomycin or to hygromycin were inserted between the fragments framing 5′ and 3′. The following ACP primers were used for the amplification:

First Allele:

Fragment 5′,
5′-iota1-5′, CACTCGAGTACCAGCCGTCTGGTGTTTG,
and
5′-iota1-3′, CAGTCGACTACAGTCTCCAGTCGCTC (3.1 kb);
fragment 3′,
3′-iota1-5′, CACTCGAGCTCAAATCCAGAGCTAAAAG,
and
3′-iota1-3′, CAGTCGACATAGTTGCAGGTAACCACC (2.5 kb).

Second Allele:

```
Fragment 5′,
5′-iota2-5′, CACTCGAGAGAGCGACTGGAGACTGTAG,
and
5′-iota2-3′, ACGTCGACAGGAAGAATGCAGAGTGACG
                        (1.8 kb);
fragment 3′,
3′-iota2-5′, CTCAGTCGACGGTGGTTACCTGCAACTATG,
and
3′-iota2-3′, CCAAGTCTCTCAACAACTGG
                          (3.8 KB).
```

Pfu turbo polymerases (Stratagene) were used for amplification of fragment 5′ of the first construction, and Herculase (Stratagene) for fragment 3′ of the second construction (30 sec at 94° C., 30 sec at 62° C. and 12 minutes at 68° C. for 40 cycles); the ACP system Expand Long Template PCR system (Roche) was used for fragment 3′ of the first construction (40 cycles in accordance with the provider's conditions, comprising an increment in the elongation time), and the ACP kit Advantage 2 PCR (Clontech) was used for fragment 5′ of the second construction (30 sec at 94° C., 30 sec at 64° C. and 4 min at 68° C.). Transfection of 30 μg of constructs linearized by Notl or Mlul was carried out as described by Bertucci, B. et al., Immunity 9, 257-65 (1998). Screening of the targeted clones of the gene was carried out by ACP on pools of 10 clones with the following primers situated outside the construction and inside the neomycin- or hygromycin-resisting gene:

First allele,
iota-5′-test, ATGACCCAAGCTCAAGACAGC
and
neo^{R}: CATAGCGTTGGCTACCCGTG in 5′
and
3′-iota2-3′ and neo^{R} reverse, CACGGGTAGCCAACG
CTATG in 3′;
Second allele,
iota-5′-test and hygro^{R}, GCTGTGTAGAAGTACTCGCC

(Expand Long Template PCR System, Roche).

For the first allele, the homologous recombination was obtained in one clone out of 518 transfectants.

For the second allele, 2 clones out of 535 were correctly targeted.

This low frequency was attributed to the use of ACP enzymes of mediocre reliability in the construction of targeting vectors. Such a specific polymorphism activity of one strain on homologous recombination has been observed in murine ES cells, where a 0.6% sequence difference turned out to result in a division in the targeting efficacy by 50. Hence, the other gene inactivations of genes carried out since then on BL2 with constructions amplified solely with Pfu turbo DNA polymerase (Stratagene), which is an enzyme whose error frequency is less than 10⁻⁴ per base pair under the amplification conditions used, have given a targeting frequency in the range of 1%-2%.

Transfection of BL2 Clones deficient in Pol Iota:

Clones deficient in Pol iota were transfected with Pol iota expression vectors, constructed by amplification of a human Pol iota cDNA of total length (McDonald, I. P. et al., Genomics, 60, 20-30 (1999)) with the following primers:

5′-iota. GCGGATCCACGACGAGGAAGACG,
and
3′-iota GCGGATCCTACGCTTTGTGCCAG

(BamHI site underlined) at the BamHI site of the pIRES puro vector (Invitrogen). Transfection and selection of the clones were carried out in accordance with conventional techniques known to persons skilled in the art, as described above.
Analysis of BL2 Clones by RT-ACP and Western Blot:

Western blots were carried out in a conventional manner with anti-Pol iota polyclonal antibodies (15-aa KLH-conjugated peptide, at C-terminal end).

It was found, in addition, that Pol iota can be further improved by techniques known to persons skilled in the art (see, in particular, the technique of Prakash) developed for the Pol eta enzyme, which, like Pol iota, has an annular domain II (“ring finger”) in which it is possible to cause mutation, by random mutagenesis of said domain II, of at least one amino acid present in this “ring finger” domain, which results in a significant increase of the activity of the enzyme mutated in this manner.

Thus, the invention also relates to a kit for induction of somatic mutations such as described above, and comprising a Pol iota enzyme thus improved by random mutagenesis of its annular domain II, in order to change therein at least one amino acid and thereby improving its performances, followed by restoration of the enzyme by means such as indicated above.

Thus this system, in its different variants of realization explained above, makes it possible, in particular, to identify the mutations necessary for improving the performances of a protein of interest. For example, in the illustrated and nonlimitative case of the gene coding for insulin, placed in a promoter-activator cassette transfected in the BL2, the latter can produce numerous molecules, among which the one having the best performance will be preferentially fixed on its substrate. Then, by proceeding to sequencing of the mutated gene coding for this preferential protein and to identification, by known means, of the modified amino acids which permitted this improvement in efficacy, it is possible, by the process of the invention, to very rapidly identify the mutations whereby an insulin of greater efficacy can be obtained.

1. Process for the induction of somatic mutations in vitro, characterized in that it comprises (1) the addition to the cells to be mutated, under culturing conditions and in a medium that are suitable for said cells, of at least one combination of antibodies comprising anti-IgM antibodies selected from a combination of anti-IgM and anti-CD19 antibodies, a combination of anti-IgM and anti-CD21 antibodies and a combination of anti-IgM, anti-CD19 and anti-CD21 antibodies, while (2) said anti-IgM antibodies are biotinylated and are subjected to specific aggregation by streptavidin coupled to a support.

2. Process according to claim 1, characterized in that said support is composed, entirely or in part, of small magnetic balls.

3. Process according to either one of claims 1 or 2, characterized in that BL2 Burkitt's lymphoma cells are used as the cells to be mutated.

4. Process according to claim 1, characterized in that said somatic mutations are controlled by adjusting the amount of Pol iota DNA polymerase.

5. Process according to claim 1, characterized in that the previously inactivated Pol iota DNA polymerase is restored by transfection of pIRES vectors, leading to the expression of complete Pol iota cDNA under the control of the CMV promoter.

6. A method for carrying out in vitro hypermutagenicity tests at the protein and/or genic level comprising inducing somatic mutation using a method in accordance with claim 1.

7. A method according to claim 6, characterized in that it is applied to B lymphomas.

8. A method according to claim 7, characterized in that it is designed for the induction of mutations in BL2 Burkitt's lymphoma cells.

9. A method according to claim 8, characterized in that it aims at the induction of mutations in BL2 Burkitt's lymphoma cells in the G0/G1 phase.

10. A method for carrying out in vitro hypermutagenicity tests at the protein and/or genic level in BL2 Burkitt's lymphoma cells comprising inducing somatic mutation using a method in accordance with claim 1, characterized in that it additionally comprises the use of Pol iota DNA polymerase for adjusting the hypermutagenicity in the in vitro hypermutagenicity tests carried out at the protein and/or genic level.

11. A method according to claim 10, wherein said Pol iota DNA polymerase is an improved Pol iota DNA polymerase that is restored after change of at least one of its natural amino acids.

12. A method according to claim 11, characterized in that said Pol iota improvement is carried out by random mutagenesis of the annular domain II, with a view to replacing at least one amino acid therein.

13. Kit for the induction of somatic mutations, characterized in that it comprises means for the induction of somatic mutations, comprising at least one combination of antibodies comprising anti-IgM antibodies selected from a combination of anti-IgM and anti-CD19 antibodies, a combination of anti-IgM and anti-CD21 antibodies, and a combination of anti-IgM, anti-CD19 and anti-CD21 antibodies.

14. Kit according to claim 13, characterized in that said anti-IgM antibodies are biotinylated and have been subjected to specific aggregation by streptavidin coupled to a support, notably to small magnetic balls.

15. Kit according to either of claims 13 or 14, characterized in that it additionally comprises means for inactivation of the Pol iota gene and/or for the latter's restoration by Pol iota cDNA expression.

16. Kit according to claim 15, characterized in that it comprises an improved Pol iota DNA polymerase, i.e., one that is restored after change of at least one of its natural amino acids.

17. Kit according to claim 16, characterized in that said Pol iota DNA polymerase is a Pol iota enzyme improved by random mutagenesis of the annular domain II, carried out in order to replace at least one amino acid.

18. Kit for the induction of somatic mutations, characterized in that it comprises means for carrying out the process according to any one of claims 1 through 3.

19. A method for qualitative and/or quantitative identification of components of the mutasome comprising the use of a kit according to claim 13.

20. A method according to claim 19, characterized in that it comprises identification of the induced post-translational modifications, carried out, in particular, by identification, isolation and analysis of a component of the mutasome which appears during said induction.

21. A method according to claim 19, characterized in that it comprises the provision of at least one gene, notably a gene coding for a protein of interest, in which it is desired to induce mutations, while said gene is included in a cassette containing the promoter and activator (enhancer) of the genes coding for the heavy or light chain of the IgG's, and that said gene is transfected in lymphoma cells, in particular in BL2 Burkitt's lyrriphoma cells.

22. A method according to claim 19, characterized in that, in order to cause mutation of any given sequence, said sequence to be mutated is surrounded by the promoter and activator of the Ig's, preferably in a mutation cassette.

23. A method according to claim 19, characterized in that, in order to mutate any given sequence, this sequence is inserted by homologous recombination at the locus of the immunoglobulins, replacing all or part of the V gene of one of the two rearranged loci.
